(19) United States
(12) Patent Application Publication
(10) Pub. No.: US 2026/0112142 A1
(43) Pub. Date: Apr. 23, 2026

(54) PERSONALIZED SEGMENTATION MODEL

(71) Applicant: Elekta, Inc., Atlanta, GA (US)

(72) Inventors: Maurits Boudewijn Wolleswinkel, Amsterdam (NL); Rui Lopes, Brossard (CA); Tony Falco, La Prairie (CA); Joseph Stancanello, Gif sur Yvette (FR)

(21) Appl. No.: 18/918,636

(22) Filed: Oct. 17, 2024

(51) Int. Cl. G06V 10/26; A61N 5/10; G06V 10/774; G06V 10/82; G06V 10/94

(52) U.S. Cl. CPC G06V 10/26; A61N 5/1039; G06V 10/774; G06V 10/82; G06V 10/945; G06V 2201/03

(57) ABSTRACT

Systems and techniques may be used for generating a personalized segmentation model. For example, a method may include, using a first set of labeled image structures, to train, using processing circuitry, an initial segmentation model. The method may include sending, to a user, the initial segmentation model and selected slices from a second set of planning image structures, and receiving, from the user, adjusted selected slices, the adjusted selected slices being modified by the user. The method may include generating, using the adjusted selected slices as an input to a structure model, a set of contoured structures. The method may include modifying the initial segmentation model using the set of contoured structures via a transfer learning technique to output a personalized segmentation model for the user.

### TECHNICAL FIELD

Embodiments of the present disclosure pertain generally to medical image and artificial intelligence processing techniques. In particular, the present disclosure pertains to training a personalized segmentation model.

### BACKGROUND

Radiation therapy can involve a planning stage and a treatment stage. In the planning stage, an imager can be used to obtain internal medical images of a lesion. These images can be used such as to measure the location, size, contour, and amount of critical structures to be treated. This information can be used to establish a dose distribution, and various other irradiation parameters to irradiate the lesion while minimizing damage to surrounding healthy tissue. These images can be factored into a subsequent treatment planning stage, e.g., involving the delineation of target volumes and normal critical organs in the images. Accurately tracking the various objects, such as a tumor, healthy tissue, or other aspects of patient anatomy can be challenging, e.g., when the patient is moving (e.g., breathing), when an image can include noise, or when an image represents organs proximate to one another.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example but not by way of limitation, various embodiments discussed in the present document.

FIG. 1 is a block diagram showing aspects personalized segmentation.

FIG. 2 is a system diagram for generating a personalized segmentation model.

FIG. 3 is a flowchart showing a process for training a machine learning model.

FIG. 4 is an example training diagram for training a machine learning model.

FIG. 5 is a flowchart of exemplary operations for generating a personalized segmentation model.

FIG. 6 illustrates an exemplary radiotherapy system adapted for performing image patient state estimation processing.

FIG. 7 illustrates an exemplary image-guided radiotherapy device.

FIG. 8 illustrates a partially cut-away view of an exemplary system including a combined radiation therapy system and an imaging system, such as a nuclear magnetic resonance (MR) imaging system.

### DETAILED DESCRIPTION

In the following detailed description, reference is made to the accompanying drawings which form a part hereof, and which is shown by way of illustration-specific embodiments in which the present invention may be practiced. These embodiments, which are also referred to herein as “examples,” are described in sufficient detail to enable those skilled in the art to practice the invention, and it is to be understood that the embodiments may be combined, or that other embodiments may be utilized and that structural, logical and electrical changes may be made without departing from the scope of the present invention. The following detailed description is, therefore, not be taken in a limiting sense, and the scope of the present invention is defined by the appended claims and their equivalents.

In radiotherapy or radiosurgery, treatment planning can be performed based on medical images of a patient, such as involving a delineation of target volumes and organs in the medical images. It can be challenging to accurately track various objects of interest, such as a tumor, healthy tissue, or other aspects of patient anatomy when the patient is moving (e.g., breathing). For example, certain approaches involving 2D imaging, 2D kV projections, 2D MRI slices, etc. can be inadequate for directly measuring a changing patient state in real-time. Another approach to tracking objects of interest involves, e.g., detecting surface information, either directly or by tracking markers on a vest or a box affixed to the patient. Such an approach can assume that the surface information is correlated to internal patient state, and such an assumption can be inaccurate or unsuitable for certain situations. Another approach involves implanting markers, such as magnetically tracked markers, or using x-ray detection of radio-opaque markers. Such approaches can be unsuitable for certain patients or situations, as implantation of devices can be invasive while corresponding only to a relatively small number of points within the patient. Yet another approach involves a model such as a regression model to assist in motion monitoring. Achieving accuracy can be challenging for such an approach, especially when seeking to track motion in substantially real-time.

The present inventors have recognized the benefits of a technique that allows for a deep learning model for structure segmentation to be adapted and personalized to a user or treatment center's preferences, such as personalized for a particular contouring style. The technique can involve learning segmentation from a database of datasets segmented by expert radiation oncologists. The technique can also involve adding information from a user “segmentation style”, such as preference for inclusion or exclusion of certain data elements. For example, a particular user may tend to track a specific boundary of an organ to generate a different type of segmentation than other medical practitioners, although both target and obtain similar degrees of accuracy from the contours.

In an example, the technique can be practiced including or using system for creating personalized segmentation models using medical image data. The system can facilitate retrieving planning image structures from a database, training an initial segmentation model with these structures, and sending this model along with selected slices to a user for adjustments. In an example, the adjusted slices can be used to generate contoured structures, which modify the initial model through transfer learning, resulting in a personalized model tailored to the user. In an example, the effectiveness of the personalized model can be tested against a third set of image structures, such as to ensure certain parameters (e.g., a DICE coefficient or surface distance error) fall within a specified user variability range. The system can facilitate user interaction, e.g., including user input from a clinician, such as to adjust model outputs and iteratively update the model during clinical workflows.

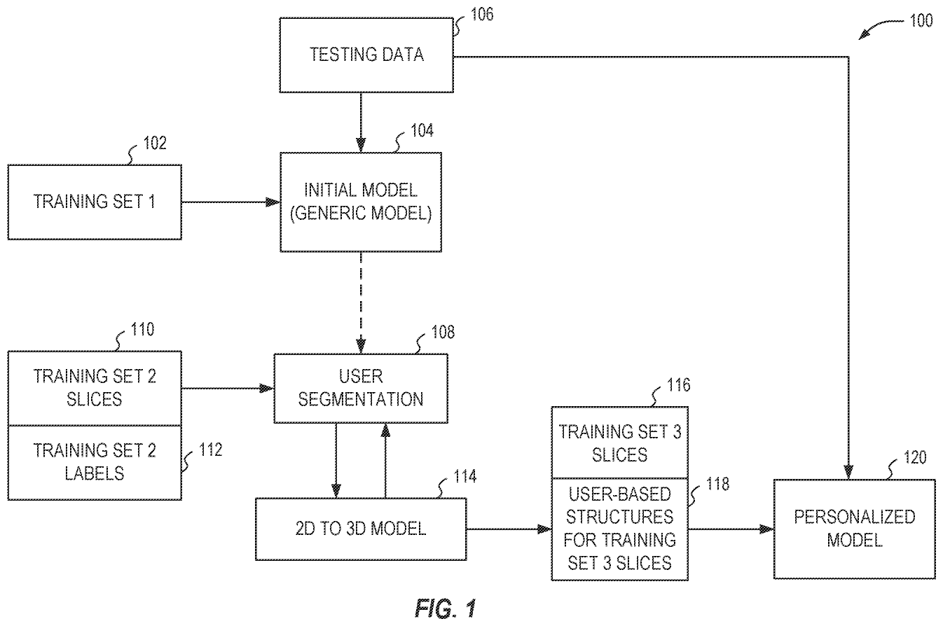

FIG. 1 is a block diagram including aspects of personalized segmentation. Generally, radiotherapy (RT) can induce a differential response to ionizing radiation of healthy and cancerous cells. In an example, objects of interest such as a lesion (also referred to herein as a target) and nearby organs at risk (OARs) can be identified during the offline treatment planning phase by means of treatment planning system (TPS). The process of identification of such objects of interest can also be referred to herein as segmentation. Such segmentation can involve a series of steps which can be manually, semi-automatic or fully automatic performed on a Computer Tomography (CT) or planning CT. Certain approaches to manual segmentation can be tedious, time-consuming process. For example, manual segmentation can involve a qualified medical professional (e.g., a radiation oncologist or dosimetrist) to provide knowledge and experience for tailoring segmentation to specific characteristics of the patient, treatment guidelines and treatment delivery (e.g. conventional RT or advanced techniques like stereotactic body RT, SBRT). Following such segmentation, RT treatments can be administered over a course of several fractions, e.g., ranging from about 3 to about 40 fractions. Where a relatively low number of fractions are administered, RT treatments can be referred to as “hypofractionated” treatments, such as involving desirable radiobiological parameters and desirable quality of life benefits to a patient under treatment. Such hypofractionated treatments can be promoted by certain conformal RT treatments such as Intensity Modulated RT (IMRT) or Volumetric Modulated Arc Therapy (VMAT) capable of achieving high conformity index as well as steep dose gradients around the target, referred to as dose fall-off. Such treatments can involve particular attention toward patient repositioning and especially the relative position of the target to nearby OARs, such as to facilitate tumor control while mitigating any potential side effects to the RT treatment.

In an example, RT can be performed including onboard imaging, e.g., using a Cone Beam Computer Tomography (CBCT) system. CBCT systems can especially benefit from efficient transfer the segmentation contours identified on the planning CT to the daily CBCT. CBCT systems can benefit from automatically segmenting targets and OARs, e.g., directly on daily CBCT. Certain approaches to at least partially automatic segmentation of a CT struggle with receiving user specific knowledge and expertise, such as to customize the segmentation process to the need of each user or class of users.

In an example, certain Artificial Intelligence (AI) techniques (e.g., deep learning (DL) techniques) can be employed to help develop a model having both generally applicability (e.g., suitable for a vast majority of users) as well as capable of embedding user specific expertise, knowledge and experience. For example, certain transfer learning (TL) approaches involving can involve a model being trained to absorb the bulk of information from large database and subsequently tailored toward specific knowledge derived by user by training specific layers of the neural network (NN) DL consists of. When prediction problems suffer from class imbalance (e.g. when only a few instances of a specific event such as small number of segmentations by a specific user are present), data augmentation techniques can be used to help reinforce the input of class imbalanced events. For such a DL approach to facilitate accurate and robust segmentation of a structure, it can be important that the DL network “has seen” a specific “segmentation style” during the training phase. This can be challenging, such as due to the limited number of cases used in the training phase, even if data collection aims at incorporating as many as input in terms of “variety” and “veracity”.

Segmentation accuracy can be characterized in terms of volume overlapping indices (e.g. the DICE index) or of surface distance measurements (e.g. considering the minimum distance for each point of a predicted segmentation surface from the corresponding ground truth surface, such as using a Hausdorff distance). For example, a DICE score can provide a global assessment of the segmentation accuracy with respect to a ground truth. Likewise, surface distance measurement can provide local accuracy information distinguishing those regions where segmentation is accurate from those where segmentation is inaccurate. Regardless, in an example an accuracy of segmentation can be evaluated by comparing the segmentation error (e.g. mean, median maximum values, etc.) to the uncertainty present in the segmentation dataset, such as a maximum linear dimension within the voxel. Achieving automatic segmentation accuracy, e.g. with a DICE coefficient higher than 85%, can be especially challenging using certain segmentation approaches. For example, such certain segmentation approaches can operate at least partially based on rule-based approach. For example, if image gradient in a region is higher than a given threshold, then a line should be automatically drawn there. The present inventors have recognized the benefits of a technique that looks at data driven DL tailored to user personalized “segmentation style”, where segmentation fine tuning allows the user to embed specific knowledge and expertise, such as to help accelerate the segmentation process.

The block diagram of FIG. 1 depicts a technique 100 for generating personalized segmentation models using deep learning (DL). Such a technique 100 can facilitate a customization and accuracy of automated medical image segmentation, e.g., including focusing on the integration of user-specific data and the transformation of two-dimensional image slices into three-dimensional structures.

In an example, Training Set 1 102 can include an initial data pool from which the initial model 104 is trained. This set can include a collection of labeled image structures that are used to teach the model the basic parameters of image segmentation. The quality and diversity of the data in Training Set 1 can be important, such that an initial model 104 has a robust baseline from which to learn.

The initial model 104 can receive the data from Training Set 1 102. The initial model 104 can serve as the generic or preliminary segmentation model before any user-specific customization. For example, the initial model 104 can perform basic segmentation tasks and establish a preliminary accuracy level that will be enhanced through subsequent user interactions and data inputs.

In an example, the technique 100 can include using testing data 106 to help evaluate an accuracy and effectiveness of the initial model 104. The testing data 196 can aid in identifying certain strengths or weaknesses of the initial model 104, such as to providing feedback that can inform downstream adjustments and improvements. For example, such testing data 106 can be used to check that the initial model 104 meets certain specified standards before it is personalized further.

In an example, the technique 100 can include user segmentation 108, e.g., involving an input via an end user, such as where the user interacts with the model by providing specific feedback or adjustments. The user segmentation 108 can facilitate certain user-specific preferences and expertise (e.g., characteristic of a particular user's segmentation style), which can help in tailoring the model more closely to the needs of the end user or a class of similar users. In an example, the technique 100 can include determining one or regions where consensus between prediction and ground truth during the training phase will be high. For regions where consensus between prediction and ground truth during the training phase will be low, the system can prompt or request certain user input such as to supply additional information on a specific set of slices to be used to train the model by means of TL and data augmentation. For example, the technique 100 can include requesting the user to check/edit certain contours in slices where, e.g., dose fall-off is expected to be relatively high or where highly dose-sensitive OARs are at or near. For example, the technique 100 can include receiving an indication of an importance of a particular slice, such that slices of relatively high importance can be identified by the user. Such slices indicated as having importance can be displayed on a user interface device, such that the user can interact with the system for positive (e.g., training data) or negative (e.g., model errors) feedback. For example, the technique 100 can include enabling a user to indicate a preference for including or excluding a particular anatomical region from training, such as enabling the user to adjust the priority of a particular anatomical region for training.

In an example, the technique 100 can include receiving training set 2 slices 110 and/or their corresponding labels (112), which can be used for further training and refining the model. The training set 2 slices 110 and labels 112 can provide additional data points that improve the learning from that of the initial model 104, such as in areas where the initial model 104 may have shown deficiencies.

The technique can involve a the 2D to 3D model 114 configured to transform two-dimensional image slices into three-dimensional structures. Such a conversion can help during circumstances where volumetric data representations provide significant advantages, such as in detailed medical imaging or complex anatomical assessments. For example, the 2D to 3D model 114 can be customized based on training set 3 slices 116, including further user feedback and adjustments. Also, user-based structures for training set 3 slices 118 can be generated from the user-modified two-dimensional training set 2 slices 110, which are then used to train the model further to establish a personalized model 120, facilitating application of a “segmentation style” characteristic of the particular end user. For example the personalized model 120 can represent a culmination of the training and customization process. The personalized model 120 can represent a refined version of the initial model 104, adjusted and improved through user feedback, additional training sets, and the conversion of 2D slices to 3D structures via the 2D to 3D model 114. In an example, the personalized model 120 can implement further feedback via the testing data 106, e.g., in a similar fashion to that of the initial model 104. In an example, personalized model 120 can be determined as acceptable once an average error (e.g. in terms of DICE coefficient or maximum surface distance) is in the range of a specified user variability.

In an example, during the user segmentation 108, the end user can be shown a series of slices from the training set 1 102, originating from a limited number of planning CTs where user variability has been identified as high. Here, a central body of prostate can be accurately segmented by certain DL methods while apex and lower base can exhibit an unacceptable variability, such as generally requiring substantial user editing. As such, the system can show the end user such slices and ask to double check the output of the segmentation from the initial model 104, with the possibility to edit those particular slices. Other slices, such as those where prostate-bladder and prostate-rectum interfaces are present and where a high dose fall-off is expected, can also be shown and requested to be double checked or edited. As such, the technique can involve retraining the initial model 104 by means of TL and data augmentation techniques, such that the personalized model 120 incorporates the experience of the particular end user.

In an example, the technique 100 can involve further testing of the personalized model 120. Once the personalized model 120 has been trained and checked, during the course of the clinical prostate workflow in the clinic, the end user can be shown the results of the predicted segmentation corresponding with an individual patient for end user's review. Where additional editing is applied by the end user, the technique 100 can involve incorporating such newly generated information to once again to retrain and check the personalized model 120. Such retraining can ensure the personalized model 120 is continually updated over multiple iterations, e.g., including new datasets from the particular end user, e.g. using techniques of reinforcement learning.

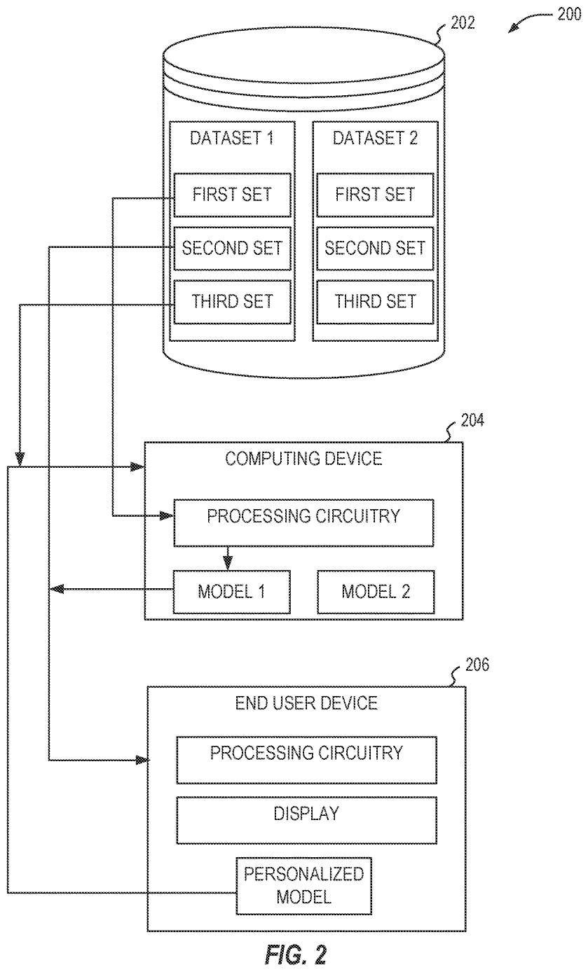

The technique 100 can integrate such aspects into a cohesive workflow (e.g., which can be performed on a system 200 as described with respect to FIG. 2) that facilitates user interaction and data-driven learning. In an example, the technique can include or use at least one variation of a U-NET model to train a network (e.g., as the initial model 104).

FIG. 2 is a system diagram for generating a personalized segmentation model. In an example, the system 200 can include or use a database 202, a computing device 204, and an end user device 206. The database 202 can include a plurality of segmented planning computer tomography (CT) datasets. In an example, the database 202 can provide a repository for medical image data. The database can be used to retrieve a first set of planning image structures which are labeled and utilized in the training of an initial segmentation model. The database can facilitate access to technically qualified and diverse datasets. In an example, the CT datasets can correspond with a prostate of each patient, and the CT datasets. For example, an individual dataset of the database 202 can be pre-segmented for the prostate and organs at risk (OARs), such as by user input of a specialist, such as a radiation oncologist. The individual dataset of the database 202 can be pre-segmented for the prostate and OARs via a plurality of different specialists (e.g., by users among a pool of expert radiation oncologists). Here, the same planning CT can be segmented by a plurality of different specialists. Comparison of the different segmentations, each corresponding with a different specialist, can be used such as to evaluate a user variability of the segmentation. In an example, the plurality of different specialists can be selected such as to form a geographically diverse demographic, e.g., expert radiation oncologists practicing in several different countries. In an example, a deep learning model can be trained by using the segmented planning CTs, e.g., using at least a portion of the datasets for training, at least a portion of the datasets, for validation and at least a portion of the datasets for testing.

The computing device 204 can include or use processing circuitry. The computing device 204 can facilitate training of an initial segmentation model, e.g., using the labeled image structures retrieved from the database. Such a model can be delivered to an end user, such as for review or adjustment. The computing device 204 can also receive user feedback, such as one or more adjusted selected slices from the end user, which can be used to generate a set of contoured structures. Such structures can be used such as to modify the initial segmentation model through a transfer learning technique and ultimately to produce a personalized segmentation model tailored to the user's specific needs and preferences.

The end user device 206 can include or use processing circuitry and a display for facilitating viewing or adjusting of the segmentation slices, sent by the computing device 204, by the end user. In an example, the end user device 206 can receive a first, initial segmentation model along with selected slices from a second set of planning image structures. The end user can modify these slices, such as by adjusting the contoured structures, adding or removing contours, or the like. The adjusted slices can be sent back to the computing device 204 for further processing, such as via a second model received or used by the computing device 204. For example, the second model can include modified data that has been derived from the first, initial model such as based on the adjustments made by the end user.

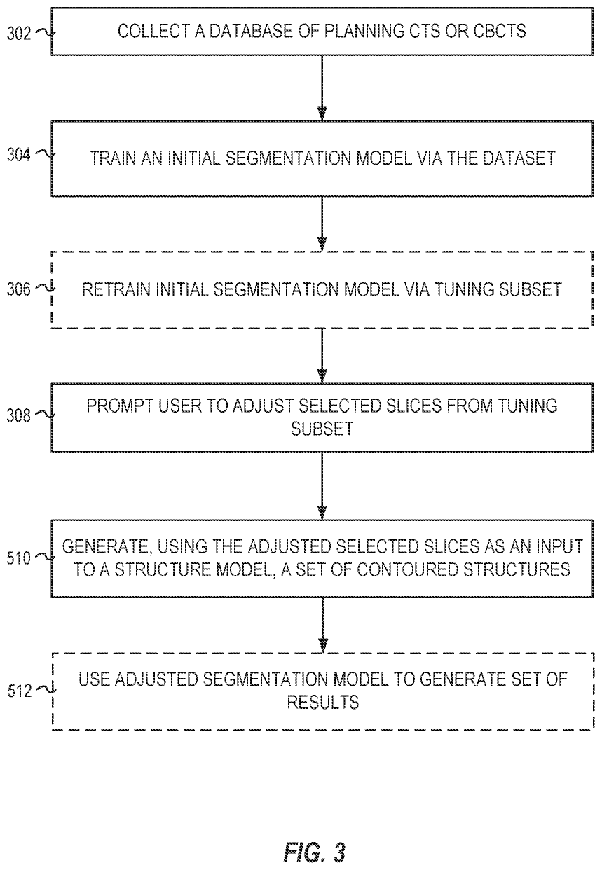

FIG. 3 is a flowchart showing a process for training a machine learning model.

At 302, a process 300 for training a machine learning model for personalized segmentation can include retrieving information from a database of planning CTs or CBCTs for a specified RT workflow (e.g. prostate, breast, etc.). In an example, the database can include a training subset, a validation subset, and a tuning subset. In an example, the validation subset can represent an “expert ground truth”. Images in the database can be, e.g., labelled by expert radiation oncologists. In an example, the process 300 can include facilitating segmenting of at least one additional subset of the database via a pool of expert radiation oncologists, such as for determining at least one segmentation user variability measurement.

At 304, the process 300 can involve training an initial segmentation model via the dataset. For example, the initial segmentation model can be configured to as disease or organ specific. The trained initial segmentation model can be tested, e.g., using the validation subset, to determine an accuracy of the initial segmentation model. Based on such a determined accuracy, the technique can involve 300 determining an acceptability of the model based on whether a specified metric (e.g., a DICE coefficient, surface distance error, etc.) is within the user variability (previously determined based on the at least one segmentation user variability measurement).

Optionally, at 306, the initial segmentation model can be further trained with the tuning subset. Here, the tuning subset can be taken from the training subset and can not share data with the at least one additional subset of the database. In an example, the tuning subset can not be shared with or manipulated directly by a user, but rather presented to the user via selectively proposing or presenting “key” slices of anatomy from the tuning subset, but not entirely presenting the corresponding 3D structures. For example, selected slices of the tuning subset can be presented based on specified criteria (e.g. highest user variability or highest expected dose-fall off around the target).

At 308, the process 300 can involve prompting user to adjust selected slices from the tuning subset. For example, such adjusting can be received by a 2D to 3D model configured to generate a 3D structure from selected 2D structures. For example, the technique can involve re-presenting slices from these new 3D structures to the user such as for the user to adjust and further customize. Such additional “measurements” can facilitate a convergency of the structures with the user “segmentation style”. Convergency criteria can be based on specified accuracy indices, such as based on a DICE coefficient or maximum surface distance between predicted structure segmentation and that fine-tuned by the user.

At 310, the process 300 can involve using the 2D to 3D model to generate a full set of 3D structures corresponding with the tuning subset. Such contoured, 3D structures can be used to further adjust the initial segmentation model, e.g., via TL and re-adjusting the weights of the trained model using the newly labelled datasets from the tuning subset.

Optionally, at 312, the process 300 can involve using the resulting adjusted segmentation model to generate a set of results from the validation subset to be used to compare against the accuracy and specified minimum performance claims consistent with a specified clearance of the model.

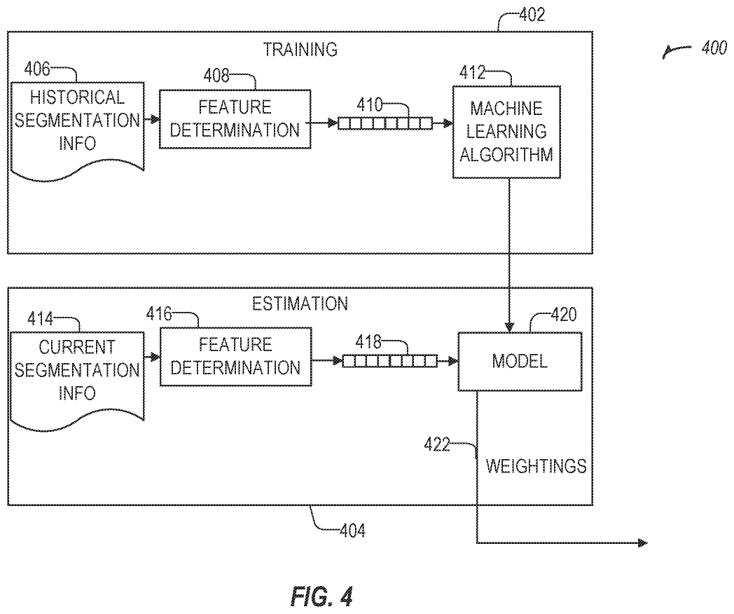

FIG. 4 illustrates an exemplary model machine learning engine 400 for use in outputting personalized segmentation information. Machine learning engine 400 utilizes a training engine 402 and an estimation engine 404. Training engine 402 inputs historical segmentation information 406 (e.g., contouring of an image via one or more technical specialists) into feature determination engine 408. The historical segmentation information 406 may be labeled to indicate a particular segmentation or a personalization of segmentation information.

Feature determination engine 408 determines one or more features 410 from this historical segmentation information 406. Stated generally, features 410 are a set of the information input and include information determined to be predictive of a particular outcome. The features 410 may be determined by hidden layers, in an example. The machine learning algorithm 412 produces a correspondence motion model 420 based upon the features 410 and the labels.

In the estimation engine 404, current segmentation information 414 (e.g., a current segmentation contouring value, an image characteristic such as a perceived border, etc.) may be input to the feature determination engine 416. Feature determination engine 416 may determine features of the current information 414 to perform automated or semiautomated image segmentation. In some examples, feature determination engines 416 and 408 are the same engine. Feature determination engine 416 produces feature vector 418, which is input into the model 420 to generate one or more criteria weightings 422. The training engine 402 may operate in an offline manner to train the model 420. The estimation engine 404, however, may be designed to operate in an online manner. It should be noted that the model 420 may be periodically updated via additional training or user feedback (e.g., additional, changed, or removed segmentation information).

The machine learning algorithm 412 may be selected from among many different potential supervised or unsupervised machine learning algorithms. Examples of supervised learning algorithms include artificial neural networks, Bayesian networks, instance-based learning, support vector machines, decision trees (e.g., Iterative Dichotomiser 3, C4.5, Classification and Regression Tree (CART), Chi-squared Automatic Interaction Detector (CHAID), and the like), random forests, linear classifiers, quadratic classifiers, k-nearest neighbor, linear regression, logistic regression, and hidden Markov models. Examples of unsupervised learning algorithms include expectation-maximization algorithms, vector quantization, and information bottleneck method. Unsupervised models may not have a training engine 402.

The model 420, once trained may output a personalized segmentation model for a user.

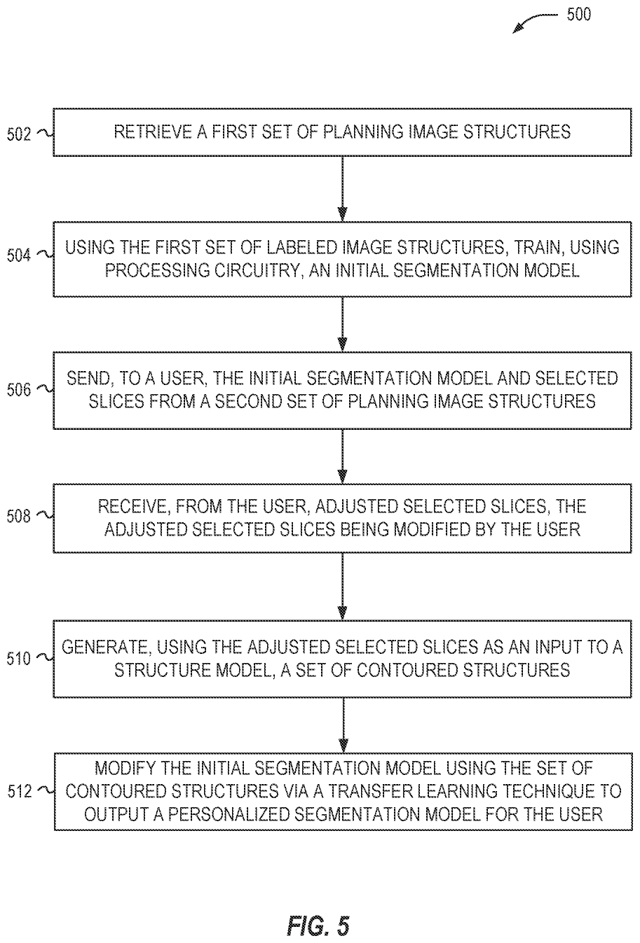

FIG. 5 illustrates a flowchart of exemplary operations for generating a personalized segmentation model, according to a technique 500.

The technique 500 may include an optional operation 502 to retrieve a first set of planning image structures. The technique 500 may include an optional an operation 504 to using the first set of planning image structures, train, using processing circuitry, an initial segmentation model. In some examples the model is pre-trained (e.g., trained and then stored for later use). The initial segmentation model may be generated using contouring by a set of clinicians of a clinic, for example when the user is a member of the clinic. In other examples, the segmentation model may be generated using contouring by experts identified, for example by a governing body (e.g., a government) or a professional organization.

The technique 500 includes an operation 506 to send, to a user, an initial segmentation model (e.g., an initial segmentation model trained using a first set of planning image structures) and selected slices from a second set of planning image structures. The technique 500 includes an operation 508 to receive, from the user, adjusted selected slices, the adjusted selected slices being modified by the user. In some examples, the adjusted selected slices may include a minimum number of slices, such as 1, 5, 10, 20, 200, etc.

The technique 500 includes an operation 510 to generate, using the adjusted selected slices as an input to a structure model, a set of contoured structures. The structure model may be trained to convert two-dimensional slices to three-dimensional structures, in some examples.

The technique 500 includes an operation 512 to modify the initial segmentation model using the set of contoured structures via a transfer learning technique to output a personalized segmentation model for the user. In some examples, the initial segmentation model or the personalized segmentation model may be specific to a particular disease or organ.

The technique 500 may include testing the personalized segmentation model, using a third set of planning image structures, for example by determining whether a metric of the personalized segmentation model is within a user variability range. In this example, the metric may include a DICE coefficient or a surface distance error.

The technique 500 may include sending, to the user, updated slices from the set of contoured structures for iteratively adjusting the updated slices. The technique 500 may include receiving, during a clinical workflow, a user edit to a segmentation generated by the personalized segmentation model. This example may include using the segmentation to iteratively update the personalized segmentation model via the transfer learning technique. Here, the technique 500 can further include receiving a response from the user, subsequent sending the updated slices, and the response can provide an indication of an acceptability of the updated slices, such as to help user-validate the iteratively adjusted slices based on, e.g., accuracy, precision, appropriateness to a specified procedure, or according to another desired characteristic.

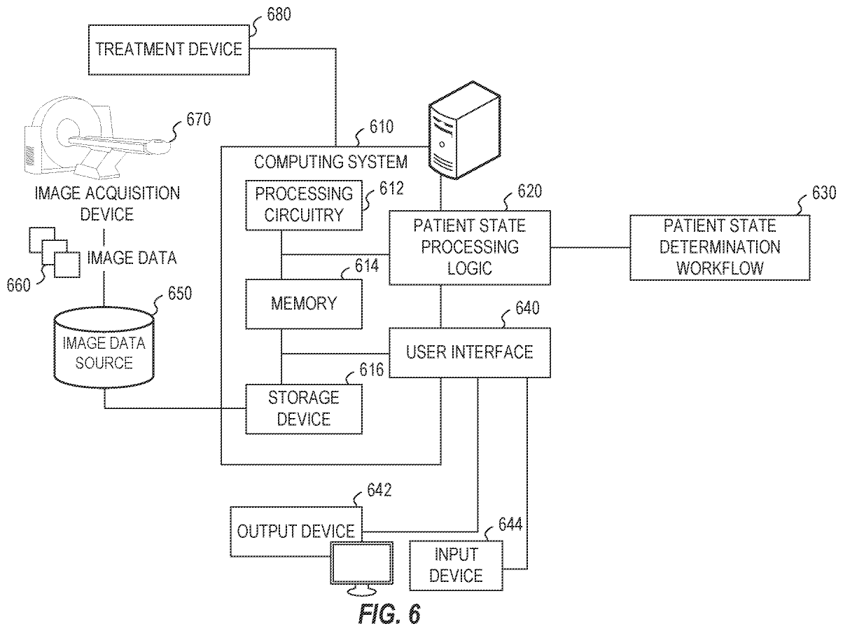

FIG. 6 illustrates an exemplary radiotherapy system adapted for using deep neural networks for real-time motion monitoring. The real-time motion monitoring may be used to determine a patient state to enable the radiotherapy system to provide radiation therapy to a patient based on specific aspects of captured medical imaging data. The radiotherapy system includes an image processing computing system 610 which hosts patient state processing logic 620. The image processing computing system 610 may be connected to a network (not shown), and such network may be connected to the Internet. For instance, a network can connect the image processing computing system 610 with one or more medical information sources (e.g., a radiology information system (RIS), a medical record system (e.g., an electronic medical record (EMR)/electronic health record (EHR) system), an oncology information system (OIS)), one or more image data sources 650, an image acquisition device 670, and a treatment device 680 (e.g., a radiation therapy device). As an example, the image processing computing system 610 can be configured to perform image patient state operations by executing instructions or data from the patient state processing logic 620, as part of operations to generate and customize radiation therapy treatment plans to be used by the treatment device 680.

The image processing computing system 610 may include processing circuitry 612, memory 614, a storage device 616, and other hardware and software-operable features such as a user interface 640, communication interface, and the like. The storage device 616 may store computer-executable instructions, such as an operating system, radiation therapy treatment plans (e.g., original treatment plans, adapted treatment plans, or the like), software programs (e.g., radiotherapy treatment plan software, artificial intelligence implementations such as deep learning models, machine learning models, and neural networks, etc.), and any other computer-executable instructions to be executed by the processing circuitry 612.

In an example, the processing circuitry 612 may include a processing device, such as one or more general-purpose processing devices such as a microprocessor, a central processing unit (CPU), a graphics processing unit (GPU), an accelerated processing unit (APU), or the like. More particularly, the processing circuitry 612 may be a complex instruction set computing (CISC) microprocessor, a reduced instruction set computing (RISC) microprocessor, a very long instruction Word (VLIW) microprocessor, a processor implementing other instruction sets, or processors implementing a combination of instruction sets. The processing circuitry 612 may also be implemented by one or more special-purpose processing devices such as an application specific integrated circuit (ASIC), a field programmable gate array (FPGA), a digital signal processor (DSP), a System on a Chip (SoC), or the like. As would be appreciated by those skilled in the art, in some examples, the processing circuitry 612 may be a special-purpose processor, rather than a general-purpose processor. The processing circuitry 612 may include one or more known processing devices, such as a microprocessor from the Pentium™, Core™, Xeon™, or Itanium® family manufactured by Intel™, the Turion™, Athlon™, Sempron™, Opteron™, FX™, Phenom™ family manufactured by AMD™, or any of various processors manufactured by Sun Microsystems. The processing circuitry 612 may also include graphical processing units such as a GPU from the GeForce®, Quadro®, Tesla® family manufactured by Nvidia™, GMA, Iris™ family manufactured by Intel™, or the Radeon™ family manufactured by AMD™. The processing circuitry 612 may also include accelerated processing units such as the Xeon Phi™ family manufactured by Intel™. The disclosed embodiments are not limited to any type of processor(s) otherwise configured to meet the computing demands of identifying, analyzing, maintaining, generating, and/or providing large amounts of data or manipulating such data to perform the methods disclosed herein. In addition, the term “processor” may include more than one processor, for example, a multi-core design or a plurality of processors each having a multi-core design. The processing circuitry 612 can execute sequences of computer program instructions, stored in memory 614, and accessed from the storage device 616, to perform various operations, processes, methods that will be explained in greater detail below.

The memory 614 may comprise read-only memory (ROM), a phase-change random access memory (PRAM), a static random access memory (SRAM), a flash memory, a random access memory (RAM), a dynamic random access memory (DRAM) such as synchronous DRAM (SDRAM), an electrically erasable programmable read-only memory (EEPROM), a static memory (e.g., flash memory, flash disk, static random access memory) as well as other types of random access memories, a cache, a register, a compact disc read-only memory (CD-ROM), a digital versatile disc (DVD) or other optical storage, a cassette tape, other magnetic storage device, or any other non-transitory medium that may be used to store information including image, data, or computer executable instructions (e.g., stored in any format) capable of being accessed by the processing circuitry 612, or any other type of computer device. For instance, the computer program instructions can be accessed by the processing circuitry 612, read from the ROM, or any other suitable memory location, and loaded into the RAM for execution by the processing circuitry 612.

The storage device 616 may constitute a drive unit that includes a machine-readable medium on which is stored one or more sets of instructions and data structures (e.g., software) embodying or utilized by any one or more of the methodologies or functions described herein (including, in various examples, the patient state processing logic 620 and the user interface 640). The instructions may also reside, completely or at least partially, within the memory 614 and/or within the processing circuitry 612 during execution thereof by the image processing computing system 610, with the memory 614 and the processing circuitry 612 also constituting machine-readable media.

The memory device 614 or the storage device 616 may constitute a non-transitory computer-readable medium. For example, the memory device 614 or the storage device 616 may store or load instructions for one or more software applications on the computer-readable medium. Software applications stored or loaded with the memory device 614 or the storage device 616 may include, for example, an operating system for common computer systems as well as for software-controlled devices. The image processing computing system 610 may also operate a variety of software programs comprising software code for implementing the patient state processing logic 620 and the user interface 640. Further, the memory device 614 and the storage device 616 may store or load an entire software application, part of a software application, or code or data that is associated with a software application, which is executable by the processing circuitry 612. In a further example, the memory device 614 or the storage device 616 may store, load, or manipulate one or more radiation therapy treatment plans, imaging data, patient state data, dictionary entries, artificial intelligence model data, labels and mapping data, etc. It is contemplated that software programs may be stored not only on the storage device 616 and the memory 614 but also on a removable computer medium, such as a hard drive, a computer disk, a CD-ROM, a DVD, a HD, a Blu-Ray DVD, USB flash drive, a SD card, a memory stick, or any other suitable medium; such software programs may also be communicated or received over a network.

Although not depicted, the image processing computing system 610 may include a communication interface, network interface card, and communications circuitry. An example communication interface may include, for example, a network adaptor, a cable connector, a serial connector, a USB connector, a parallel connector, a high-speed data transmission adaptor (e.g., such as fiber, USB 3.0, thunderbolt, and the like), a wireless network adaptor (e.g., such as a IEEE 802.11/Wi-Fi adapter), a telecommunication adapter (e.g., to communicate with 3G, 4G/LTE, and 5G, networks and the like), and the like. Such a communication interface may include one or more digital and/or analog communication devices that permit a machine to communicate with other machines and devices, such as remotely located components, via a network. The network may provide the functionality of a local area network (LAN), a wireless network, a cloud computing environment (e.g., software as a service, platform as a service, infrastructure as a service, etc.), a client-server, a wide area network (WAN), and the like. For example, network may be a LAN or a WAN that may include other systems (including additional image processing computing systems or image-based components associated with medical imaging or radiotherapy operations).

In an example, the image processing computing system 610 may obtain image data 660 from the image data source 650, for hosting on the storage device 616 and the memory 614. In an example, the software programs operating on the image processing computing system 610 may convert medical images of one format (e.g., MRI) to another format (e.g., CT), such as by producing synthetic images, such as a pseudo-CT image. In another example, the software programs may register or associate a patient medical image (e.g., a CT image or an MR image) with that patient's dose distribution of radiotherapy treatment (e.g., also represented as an image) so that corresponding image voxels and dose voxels are appropriately associated. In yet another example, the software programs may substitute functions of the patient images such as signed distance functions or processed versions of the images that emphasize some aspect of the image information. Such functions might emphasize edges or differences in voxel textures, or other structural aspects. In another example, the software programs may visualize, hide, emphasize, or de-emphasize some aspect of anatomical features, patient measurements, patient state information, or dose or treatment information, within medical images. The storage device 616 and memory 614 may store and host data to perform these purposes, including the image data 660, patient data, and other data required to create and implement a radiation therapy treatment plan and associated patient state estimation operations.

The processing circuitry 612 may be communicatively coupled to the memory 614 and the storage device 616, and the processing circuitry 612 may be configured to execute computer executable instructions stored thereon from either the memory 614 or the storage device 616. The processing circuitry 612 may execute instructions to cause medical images from the image data 660 to be received or obtained in memory 614, and processed using the patient state processing logic 620. For example, the image processing computing system 610 may receive image data 660 from the image acquisition device 670 or image data sources 650 via a communication interface and network to be stored or cached in the storage device 616. The processing circuitry 612 may also send or update medical images stored in memory 614 or the storage device 616 via a communication interface to another database or data store (e.g., a medical facility database). In some examples, one or more of the systems may form a distributed computing/simulation environment that uses a network to collaboratively perform the embodiments described herein. In addition, such network may be connected to internet to communicate with servers and clients that reside remotely on the internet.

In further examples, the processing circuitry 612 may utilize software programs (e.g., a treatment planning software) along with the image data 660 and other patient data to create a radiation therapy treatment plan. In an example, the image data 660 may include 2D or 3D images, such as from a CT or MR. In addition, the processing circuitry 612 may utilize deep neural networks to generate an estimated patient state, for example a first deep neural network to receive an input partial measurement and a 4D patient model and output a patient state, and a second deep neural network to use the patient state and a reference image (e.g., a selected image or slice from the 4D patient model) to generate a DVF for deforming the 4D patient model based on the input partial measurement.

Further, such software programs may utilize patient state processing logic 620 to implement a patient state determination workflow 630. The processing circuitry 612 may subsequently then transmit the executable radiation therapy treatment plan via a communication interface and the network to the treatment device 680, where the radiation therapy plan will be used to treat a patient with radiation via the treatment device, consistent with results of the patient state determination workflow 630. Other outputs and uses of the software programs and the patient state determination workflow 630 may occur with use of the image processing computing system 610.

As discussed herein (e.g., with reference to the patient state determination discussed herein), the processing circuitry 612 may execute a software program that invokes the patient state processing logic 620 to implement functions including deep neural networks.

In an example, the image data 660 may include one or more MRI images (e.g., 2D MRI, 3D MRI, 2D streaming MRI, 4D MRI, 4D volumetric MRI, 4D cine MRI, etc.), functional MRI images (e.g., fMRI, DCE-MRI, diffusion MRI), Computed Tomography (CT) images (e.g., 2D CT, Cone beam CT, 3D CT, 4D CT), ultrasound images (e.g., 2D ultrasound, 3D ultrasound, 4D ultrasound), Positron Emission Tomography (PET) images, X-ray images, fluoroscopic images, radiotherapy portal images, Single-Photo Emission Computed Tomography (SPECT) images, computer generated synthetic images (e.g., pseudo-CT images) and the like. Further, the image data 660 may also include or be associated with medical image processing data, for instance, training images, and ground truth images, contoured images, and dose images. In an example, the image data 660 may be received from the image acquisition device 670 and stored in one or more of the image data sources 650 (e.g., a Picture Archiving and Communication System (PACS), a Vendor Neutral Archive (VNA), a medical record or information system, a data warehouse, etc.). Accordingly, the image acquisition device 670 may comprise a MRI imaging device, a CT imaging device, a PET imaging device, an ultrasound imaging device, a fluoroscopic device, a SPECT imaging device, an integrated Linear Accelerator and MRI imaging device, or other medical imaging devices for obtaining the medical images of the patient. The image data 660 may be received and stored in any type of data or any type of format (e.g., in a Digital Imaging and Communications in Medicine (DICOM) format) that the image acquisition device 670 and the image processing computing system 610 may use to perform operations consistent with the disclosed embodiments.

In an example, the image acquisition device 670 may be integrated with the treatment device 680 as a single apparatus (e.g., a MRI device combined with a linear accelerator, also referred to as an “MR-linac”, as shown and described in FIG. 3 below). Such an MR-linac can be used, for example, to precisely determine a location of a target organ or a target tumor in the patient, so as to direct radiation therapy accurately according to the radiation therapy treatment plan to a predetermined target. For instance, a radiation therapy treatment plan may provide information about a particular radiation dose to be applied to each patient. The radiation therapy treatment plan may also include other radiotherapy information, such as beam angles, dose-histogram-volume information, the number of radiation beams to be used during therapy, the dose per beam, and the like.

The image processing computing system 610 may communicate with an external database through a network to send/receive a plurality of various types of data related to image processing and radiotherapy operations. For example, an external database may include machine data that is information associated with the treatment device 680, the image acquisition device 670, or other machines relevant to radiotherapy or medical procedures. Machine data information may include radiation beam size, arc placement, beam on and off time duration, machine parameters, segments, multi-leaf collimator (MLC) configuration, gantry speed, MRI pulse sequence, and the like. The external database may be a storage device and may be equipped with appropriate database administration software programs. Further, such databases or data sources may include a plurality of devices or systems located either in a central or a distributed manner.

The image processing computing system 610 can collect and obtain data, and communicate with other systems, via a network using one or more communication interfaces, which are communicatively coupled to the processing circuitry 612 and the memory 614. For instance, a communication interface may provide communication connections between the image processing computing system 610 and radiotherapy system components (e.g., permitting the exchange of data with external devices). For instance, the communication interface may in some examples have appropriate interfacing circuitry from an output device 642 or an input device 644 to connect to the user interface 640, which may be a hardware keyboard, a keypad, or a touch screen through which a user may input information into the radiotherapy system.

As an example, the output device 642 may include a display device which outputs a representation of the user interface 640 and one or more aspects, visualizations, or representations of the medical images. The output device 642 may include one or more display screens that display medical images, interface information, treatment planning parameters (e.g., contours, dosages, beam angles, labels, maps, etc.) treatment plans, a target, localizing a target or tracking a target, patient state estimations (e.g., a 3D image), or any related information to the user. The input device 644 connected to the user interface 640 may be a keyboard, a keypad, a touch screen or any type of device that a user may input information to the radiotherapy system. Alternatively, the output device 642, the input device 644, and features of the user interface 640 may be integrated into a single device such as a smartphone or tablet computer, e.g., Apple iPad®, Lenovo Thinkpad®, Samsung Galaxy®, etc.

Furthermore, any and all components of the radiotherapy system may be implemented as a virtual machine (e.g., via VMWare, Hyper-V, and the like virtualization platforms). For instance, a virtual machine can be software that functions as hardware. Therefore, a virtual machine can include at least one or more virtual processors, one or more virtual memories, and one or more virtual communication interfaces that together function as hardware. For example, the image processing computing system 610, the image data sources 650, or like components, may be implemented as a virtual machine or within a cloud-based virtualization environment.

The patient state processing logic 620 or other software programs may cause the computing system to communicate with the image data sources 650 to read images into memory 614 and the storage device 616, or store images or associated data from the memory 614 or the storage device 616 to and from the image data sources 650. For example, the image data source 650 may be configured to store and provide a plurality of images (e.g., 3D MRI, 4D MRI, 2D MRI slice images, CT images, 2D Fluoroscopy images, X-ray images, raw data from MR scans or CT scans, Digital Imaging and Communications in Medicine (DICOM) metadata, etc.) that the image data source 650 hosts, from image sets in image data 660 obtained from one or more patients via the image acquisition device 670. The image data source 650 or other databases may also store data to be used by the patient state processing logic 620 when executing a software program that performs patient state estimation operations, or when creating radiation therapy treatment plans. Further, various databases may store the data produced by the preliminary motion model (such as the dictionary), the correspondence motion model, or machine learning models, including the network parameters constituting the model learned by the network and the resulting predicted data. The image processing computing system 610 thus may obtain and/or receive the image data 660 (e.g., 2D MRI slice images, CT images, 2D Fluoroscopy images, X-ray images, 3D MRI images, 4D MRI images, etc.) from the image data source 650, the image acquisition device 670, the treatment device 680 (e.g., a MRI-Linac), or other information systems, in connection with performing image patient state estimation as part of treatment or diagnostic operations.

The image acquisition device 670 can be configured to acquire one or more images of the patient's anatomy for a region of interest (e.g., a target organ, a target tumor or both). Each image, typically a 2D image or slice, can include one or more parameters (e.g., a 2D slice thickness, an orientation, and a location, etc.). In an example, the image acquisition device 670 can acquire a 2D slice in any orientation. For example, an orientation of the 2D slice can include a sagittal orientation, a coronal orientation, or an axial orientation. The processing circuitry 612 can adjust one or more parameters, such as the thickness and/or orientation of the 2D slice, to include the target organ and/or target tumor. In an example, 2D slices can be determined from information such as a 3D MRI volume. Such 2D slices can be acquired by the image acquisition device 670 in “real-time” while a patient is undergoing radiation therapy treatment, for example, when using the treatment device 680 (with “real-time” meaning acquiring the data in 10 milliseconds or less). In another example for some applications, real-time may include a timeframe within (e.g., up to) 100 or 300 milliseconds. In an example, real-time may include a time period fast enough for a clinical problem being solved by techniques described herein. In this example, real-time may vary depending on target speed, radiotherapy margins, lag, response time of a treatment device, etc.

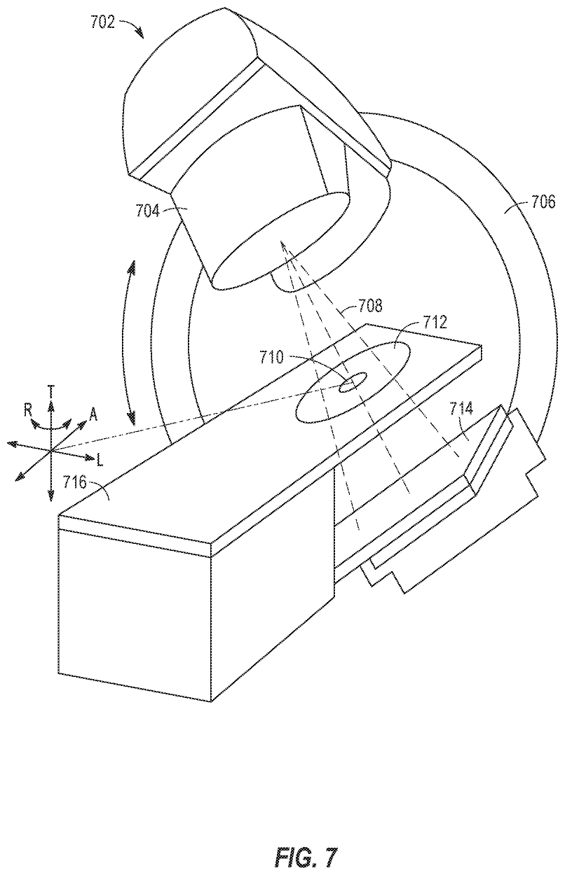

FIG. 7 illustrates an exemplary image-guided radiotherapy device 702, that includes include a radiation source, such as an X-ray source or a linear accelerator, a couch 716, an imaging detector 714, and a radiation therapy output 704. The radiation therapy device 702 may be configured to emit a radiation beam 708 to provide therapy to a patient. The radiation therapy output 704 can include one or more attenuators or collimators, such as a multi-leaf collimator (MLC).

As an example, a patient may be positioned in a region 712, supported by the treatment couch 716 to receive a radiation therapy dose according to a radiation therapy treatment plan (e.g., a treatment plan generated by the radiotherapy system of FIG. 2). The radiation therapy output 704 can be mounted or attached to a gantry 706 or other mechanical support. One or more chassis motors (not shown) may rotate the gantry 706 and the radiation therapy output 704 around couch 716 when the couch 716 is inserted into the treatment area. In an example, gantry 706 may be continuously rotatable around couch 716 when the couch 716 is inserted into the treatment area. In another example, gantry 706 may rotate to a predetermined position when the couch 716 is inserted into the treatment area. For example, the gantry 706 can be configured to rotate the therapy output 704 around an axis (“A”). Both the couch 716 and the radiation therapy output 704 can be independently moveable to other positions around the patient, such as moveable in transverse direction (“T”), moveable in a lateral direction (“L”), or as rotation about one or more other axes, such as rotation about a transverse axis (indicated as “R”). A controller communicatively connected to one or more actuators (not shown) may control the couch 716 movements or rotations in order to properly position the patient in or out of the radiation beam 708 according to a radiation therapy treatment plan. As both the couch 716 and the gantry 706 are independently moveable from one another in multiple degrees of freedom, which allows the patient to be positioned such that the radiation beam 708 precisely can target the tumor.

The coordinate system (including axes A, T, and L) shown in FIG. 7 can have an origin located at an isocenter 710. The isocenter can be defined as a location where the central axis of the radiation therapy beam 708 intersects the origin of a coordinate axis, such as to deliver a prescribed radiation dose to a location on or within a patient. Alternatively, the isocenter 710 can be defined as a location where the central axis of the radiation therapy beam 708 intersects the patient for various rotational positions of the radiation therapy output 704 as positioned by the gantry 706 around the axis A.

Gantry 706 may also have an attached imaging detector 714. The imaging detector 714 is preferably located opposite to the radiation source (output 704), and in an example, the imaging detector 714 can be located within a field of the therapy beam 708.

The imaging detector 714 can be mounted on the gantry 706 preferably opposite the radiation therapy output 704, such as to maintain alignment with the therapy beam 708. The imaging detector 714 rotating about the rotational axis as the gantry 706 rotates. In an example, the imaging detector 714 can be a flat panel detector (e.g., a direct detector or a scintillator detector). In this manner, the imaging detector 714 can be used to monitor the therapy beam 708 or the imaging detector 714 can be used for imaging the patient's anatomy, such as portal imaging. The control circuitry of radiation therapy device 702 may be integrated within the radiotherapy system or remote from it.

In an illustrative example, one or more of the couch 716, the therapy output 704, or the gantry 706 can be automatically positioned, and the therapy output 704 can establish the therapy beam 708 according to a specified dose for a particular therapy delivery instance. A sequence of therapy deliveries can be specified according to a radiation therapy treatment plan, such as using one or more different orientations or locations of the gantry 706, couch 716, or therapy output 704. The therapy deliveries can occur sequentially, but can intersect in a desired therapy locus on or within the patient, such as at the isocenter 710. A prescribed cumulative dose of radiation therapy can thereby be delivered to the therapy locus while damage to tissue nearby the therapy locus can be reduced or avoided.

Thus, FIG. 7 specifically illustrates an example of a radiation therapy device 702 operable to provide radiotherapy treatment to a patient, with a configuration where a radiation therapy output can be rotated around a central axis (e.g., an axis “A”). Other radiation therapy output configurations can be used. For example, a radiation therapy output can be mounted to a robotic arm or manipulator having multiple degrees of freedom. In yet another example, the therapy output can be fixed, such as located in a region laterally separated from the patient, and a platform supporting the patient can be used to align a radiation therapy isocenter with a specified target locus within the patient. In another example, a radiation therapy device can be a combination of a linear accelerator and an image acquisition device. In some examples, the image acquisition device may be an MRI, an X-ray, a CT, a CBCT, a spiral CT, a PET, a SPECT, an optical tomography, a fluorescence imaging, ultrasound imaging, an MR-linac, or radiotherapy portal imaging device, etc., as would be recognized by one of ordinary skill in the art.

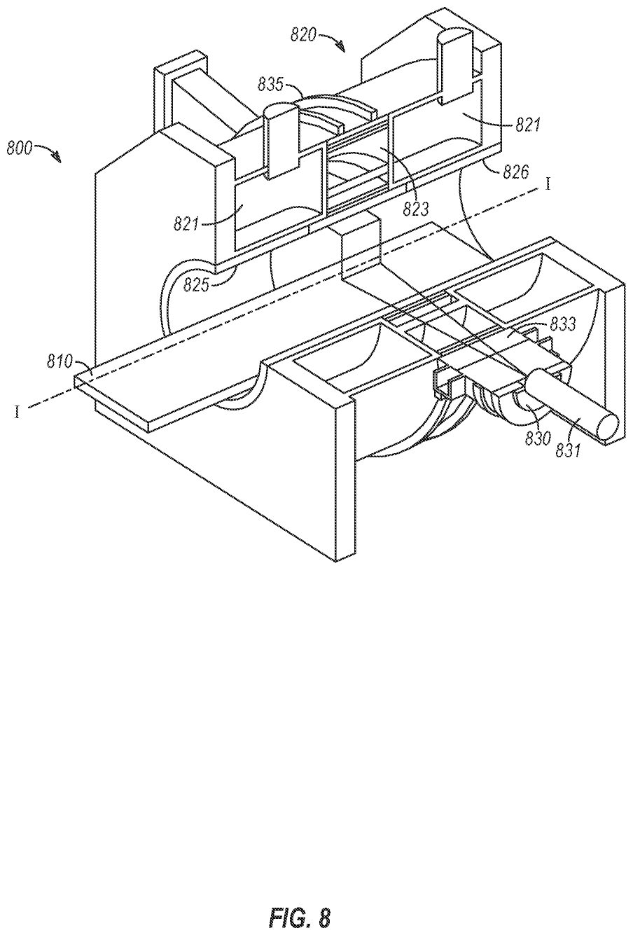

FIG. 8 depicts an exemplary radiation therapy system 800 (e.g., known in the art as a MR-Linac) that can include combining a radiation therapy device 702 and an imaging system, such as a nuclear magnetic resonance (MR) imaging system consistent with the disclosed embodiments. As shown, system 800 may include a couch 810, an image acquisition device 820, and a radiation delivery device 830. System 800 delivers radiation therapy to a patient in accordance with a radiotherapy treatment plan. In some embodiments, image acquisition device 820 may correspond to image acquisition device 670 in FIG. 6 that may acquire images.

Couch 810 may support a patient (not shown) during a treatment session. In some implementations, couch 810 may move along a horizontal, translation axis (labelled “I”), such that couch 810 may move the patient resting on couch 810 into or out of system 800. Couch 810 may also rotate around a central vertical axis of rotation, transverse to the translation axis. To allow such movement or rotation, couch 810 may have motors (not shown) enabling the couch to move in various directions and to rotate along various axes. A controller (not shown) may control these movements or rotations in order to properly position the patient according to a treatment plan.

In some embodiments, image acquisition device 820 may include an MRI machine used to acquire 2D or 3D MRI images of the patient before, during, or after a treatment session. Image acquisition device 820 may include a magnet 821 for generating a primary magnetic field for magnetic resonance imaging. The magnetic field lines generated by operation of magnet 821 may run substantially parallel to the central translation axis I. Magnet 821 may include one or more coils with an axis that runs parallel to the translation axis I. In some embodiments, the one or more coils in magnet 821 may be spaced such that a central window 823 of magnet 821 is free of coils. In other embodiments, the coils in magnet 821 may be thin enough or of a reduced density such that they are substantially transparent to radiation of the wavelength generated by radiotherapy device 830. Image acquisition device 820 may also include one or more shielding coils, which may generate a magnetic field outside magnet 821 of approximately equal magnitude and opposite polarity in order to cancel or reduce any magnetic field outside of magnet 821. As described below, radiation source 831 of radiotherapy device 830 may be positioned in the region where the magnetic field is cancelled, at least to a first order, or reduced.

Image acquisition device 820 may also include two gradient coils 825 and 826, which may generate a gradient magnetic field that is superposed on the primary magnetic field. Coils 825 and 826 may generate a gradient in the resultant magnetic field that allows spatial encoding of the protons so that their position can be determined. Gradient coils 825 and 826 may be positioned around a common central axis with the magnet 821, and may be displaced along that central axis. The displacement may create a gap, or window, between coils 825 and 826. In the embodiments where magnet 821 also includes a central window 823 between coils, the two windows may be aligned with each other.

Image acquisition is used to track tumor movement. At times, internal or external surrogates may be used. However, implanted seeds may move from their initial positions or become dislodged during radiation therapy treatment. Also, using surrogates assumes there is a correlation between tumor motion and the displacement of the external surrogate. However, there may be phase shifts between external surrogates and tumor motion, and their positions may frequently lose correlation over time. It is known that there may be mismatches between tumor and surrogates upward of 9 mm. Further, any deformation of the shape of a tumor is unknown during tracking.

An advantage of magnetic resonance imaging (MRI) is in the superior soft tissue contrast that is provided to visualize the tumor in more detail. Using a plurality of intrafractional MR images allows the determination of both shape and position (e.g., centroid) of a tumor. In addition, MRI images improve any manual contouring performed by, for example, a radiation oncologist, even when auto-contouring software (e.g., ABAS®) is utilized. This is because of the high contrast between the tumor target and the background region provided by MR images.

Another advantage of using an MR-Linac system is that a treatment beam can be continuously on and thereby executing intrafractional tracking of the target tumor. For instance, optical tracking devices or stereoscopic x-ray fluoroscopy systems can detect tumor position at 30 Hz by using tumor surrogates. With MRI, the imaging acquisition rates are faster (e.g., 3-6 fps). Therefore, the centroid position of the target may be determined, artificial intelligence (e.g., neural network) software can predict a future target position. An added advantage of intrafractional tracking by using an MR-Linac is that the by being able to predict a future target location, the leaves of the multi-leaf collimator (MLC) will be able to conform to the target contour a its predicted future position. Thus, predicting future tumor position using MRI occurs at the same rate as imaging frequency during tracking. By being able to track the movement of a target tumor clearly using detailed MRI imaging allows for the delivery of a highly conformal radiation dose to the moving target.

In some embodiments, image acquisition device 820 may be an imaging device other than an MRI, such as an X-ray, a CT, a CBCT, a spiral CT, a PET, a SPECT, an optical tomography, a fluorescence imaging, ultrasound imaging, or radiotherapy portal imaging device, etc. As would be recognized by one of ordinary skill in the art, the above description of image acquisition device 820 concerns certain embodiments and is not intended to be limiting.

Radiotherapy device 830 may include the source of radiation 831, such as an X-ray source or a linear accelerator, and a multi-leaf collimator (MLC) 833. Radiotherapy device 830 may be mounted on a chassis 835. One or more chassis motors (not shown) may rotate chassis 835 around couch 810 when couch 810 is inserted into the treatment area. In an embodiment, chassis 835 may be continuously rotatable around couch 810, when couch 810 is inserted into the treatment area. Chassis 835 may also have an attached radiation detector (not shown), preferably located opposite to radiation source 831 and with the rotational axis of chassis 835 positioned between radiation source 831 and the detector. Further, device 830 may include control circuitry (not shown) used to control, for example, one or more of couch 810, image acquisition device 820, and radiotherapy device 830. The control circuitry of radiotherapy device 830 may be integrated within system 800 or remote from it.

During a radiotherapy treatment session, a patient may be positioned on couch 810. System 800 may then move couch 810 into the treatment area defined by magnetic coils 821, 825, 826, and chassis 835. Control circuitry may then control radiation source 831, MLC 833, and the chassis motor(s) to deliver radiation to the patient through the window between coils 825 and 826 according to a radiotherapy treatment plan.

The above detailed description includes references to the accompanying drawings, which form a part of the detailed description. The drawings show, by way of illustration but not by way of limitation, specific embodiments in which the invention can be practiced. These embodiments are also referred to herein as “examples. ” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a,” “an,” “the,” and “said” are used when introducing elements of aspects of the invention or in the embodiments thereof, as is common in patent documents, to include one or more than one or more of the elements, independent of any other instances or usages of “at least one” or “one or more. ” In this document, the term “or” is used to refer to a nonexclusive or, such that “A or B” includes “A but not B,” “B but not A,” and “A and B,” unless otherwise indicated.

In the appended claims, the terms “including” and “in which” are used as the plain-English equivalents of the respective terms “comprising” and “wherein. ” Also, in the following claims, the terms “comprising,” “including,” and “having” are intended to be open-ended to mean that there may be additional elements other than the listed elements, such that after such a term (e.g., comprising, including, having) in a claim are still deemed to fall within the scope of that claim. Moreover, in the following claims, the terms “first,” “second,” and “third,” etc., are used merely as labels, and are not intended to impose numerical requirements on their objects.

The present invention also relates to a computing system adapted, configured, or operated for performing the operations herein. This system may be specially constructed for the required purposes, or it may comprise a general purpose computer selectively activated or reconfigured by a computer program (e.g., instructions, code, etc.) stored in the computer. The order of execution or performance of the operations in embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

In view of the above, it will be seen that the several objects of the invention are achieved and other advantageous results attained. Having described aspects of the invention in detail, it will be apparent that modifications and variations are possible without departing from the scope of aspects of the invention as defined in the appended claims. As various changes could be made in the above constructions, products, and methods without departing from the scope of aspects of the invention, it is intended that all matter contained in the above description and shown in the accompanying drawings shall be interpreted as illustrative and not in a limiting sense.

The above description is intended to be illustrative, and not restrictive. For example, the above-described examples (or one or more aspects thereof) may be used in combination with each other. In addition, many modifications may be made to adapt a particular situation or material to the teachings of the invention without departing from its scope. While the dimensions, types of materials and example parameters, functions, and implementations described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

Each of these non-limiting examples may stand on its own, or may be combined in various permutations or combinations with one or more of the other examples.

Example 1 is a method comprising: sending, to a user, an initial segmentation model, trained using a first set of planning image structures, and selected slices from a second set of planning image structures; receiving, from the user, adjusted selected slices, the adjusted selected slices being modified by the user; generating, using the adjusted selected slices as an input to a structure model, a set of contoured structures; and modifying, using processing circuitry, the initial segmentation model using the set of contoured structures via a transfer learning technique to output a personalized segmentation model for the user.

In Example 2, the subject matter of Example 1 includes, testing the personalized segmentation model, using a third set of planning image structures, by determining whether a metric of the personalized segmentation model is within a user variability range.

In Example 3, the subject matter of Example 2 includes, wherein the metric includes a DICE coefficient or a surface distance error.

In Example 4, the subject matter of Examples 1-3 includes, wherein the initial segmentation model is generated using contouring by a set of clinicians of a clinic, the user being a member of the clinic.

In Example 5, the subject matter of Examples 1-4 includes, sending, to the user, updated slices from the set of contoured structures for iteratively adjusting the updated slices.

In Example 6, the subject matter of Examples 1-5 includes, receiving, during a clinical workflow, a user edit to a segmentation generated by the personalized segmentation model, and using the segmentation to iteratively update the personalized segmentation model via the transfer learning technique.

In Example 7, the subject matter of Examples 1-6 includes, wherein the adjusted selected slices include ten or fewer slices.

In Example 8, the subject matter of Examples 1-7 includes, wherein the initial segmentation model and the personalized segmentation model are specific to a particular disease or organ.

In Example 9, the subject matter of Examples 1-8 includes, wherein the structure model is trained to convert two-dimensional slices to three-dimensional structures.

Example 10 is at least one machine-readable medium, including instructions, which when executed by processing circuitry, causes the processing circuitry to perform operations to: send, to a user, an initial segmentation model, trained using a first set of planning image structures, and selected slices from a second set of planning image structures; receive, from the user, adjusted selected slices, the adjusted selected slices being modified by the user; generate, using the adjusted selected slices as an input to a structure model, a set of contoured structures; and modify the initial segmentation model using the set of contoured structures via a transfer learning technique to output a personalized segmentation model for the user.

In Example 11, the subject matter of Example 10 includes, wherein the operations further cause the processing circuitry to test the personalized segmentation model, using a third set of planning image structures, by determining whether a metric of the personalized segmentation model is within a user variability range.

In Example 12, the subject matter of Example 11 includes, wherein the metric includes a DICE coefficient or a surface distance error.

In Example 13, the subject matter of Examples 10-12 includes, wherein the initial segmentation model is generated using contouring by a set of clinicians of a clinic, the user being a member of the clinic.

In Example 14, the subject matter of Examples 10-13 includes, wherein the operations further cause the processing circuitry to send, to the user, updated slices from the set of contoured structures for iteratively adjusting the updated slices.

In Example 15, the subject matter of Examples 10-14 includes, wherein the operations further cause the processing circuitry to receive, during a clinical workflow, a user edit to a segmentation generated by the personalized segmentation model, and use the segmentation to iteratively update the personalized segmentation model via the transfer learning technique.

In Example 16, the subject matter of Examples 10-15 includes, wherein the adjusted selected slices include ten or fewer slices.

In Example 17, the subject matter of Examples 10-16 includes, wherein the initial segmentation model and the personalized segmentation model are specific to a particular disease or organ.

In Example 18, the subject matter of Examples 10-17 includes, wherein the structure model is trained to convert two-dimensional slices to three-dimensional structures.

Example 19 is a system comprising: processing circuitry; and memory, including instructions, which when executed by the processing circuitry, cause the processing circuitry to: send, to a user, the initial segmentation model, trained using a first set of planning image structures, and selected slices from a second set of planning image structures; receive, from the user, adjusted selected slices, the adjusted selected slices being modified by the user; generate, using the adjusted selected slices as an input to a structure model, a set of contoured structures; and modify the initial segmentation model using the set of contoured structures via a transfer learning technique to output a personalized segmentation model for the user.

In Example 20, the subject matter of Example 19 includes, wherein the initial segmentation model and the personalized segmentation model are specific to a particular disease or organ.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1- 20.

Example 23 is a system to implement of any of Examples 1-20.

Example 24 is a method to implement of any of Examples 1-20.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

What is claimed is:

1. A method comprising:
sending, to a user, an initial segmentation model, trained using a first set of planning image structures, and selected slices from a second set of planning image structures;
receiving, from the user, adjusted selected slices, the adjusted selected slices being modified by the user;
generating, using the adjusted selected slices as an input to a structure model, a set of contoured structures; and
modifying, using processing circuitry, the initial segmentation model using the set of contoured structures via a transfer learning technique to output a personalized segmentation model for the user.

2. The method of claim 1, further comprising testing the personalized segmentation model, using a third set of planning image structures, by determining whether a metric of the personalized segmentation model is within a user variability range.

3. The method of claim 2, wherein the metric includes a DICE coefficient or a surface distance error.

4. The method of claim 1, wherein the initial segmentation model is generated using contouring by a set of clinicians of a clinic, the user being a member of the clinic.

5. The method of claim 1, further comprising, sending, to the user, updated slices from the set of contoured structures for iteratively adjusting the updated slices.

6. The method of claim 1, further comprising, receiving, during a clinical workflow, a user edit to a segmentation generated by the personalized segmentation model, and using the segmentation to iteratively update the personalized segmentation model via the transfer learning technique.

7. The method of claim 1, wherein the adjusted selected slices include ten or fewer slices.

8. The method of claim 1, wherein the initial segmentation model and the personalized segmentation model are specific to a particular disease or organ.

9. The method of claim 1, wherein the structure model is trained to convert two-dimensional slices to three-dimensional structures.

10. At least one machine-readable medium, including instructions, which when executed by processing circuitry, causes the processing circuitry to perform operations to:
send, to a user, an initial segmentation model, trained using a first set of planning image structures, and selected slices from a second set of planning image structures;
receive, from the user, adjusted selected slices, the adjusted selected slices being modified by the user;
generate, using the adjusted selected slices as an input to a structure model, a set of contoured structures; and
modify the initial segmentation model using the set of contoured structures via a transfer learning technique to output a personalized segmentation model for the user.

11. The at least one machine-readable medium of claim 10, wherein the operations further cause the processing circuitry to test the personalized segmentation model, using a third set of planning image structures, by determining whether a metric of the personalized segmentation model is within a user variability range.

12. The at least one machine-readable medium of claim 11, wherein the metric includes a DICE coefficient or a surface distance error.

13. The at least one machine-readable medium of claim 10, wherein the initial segmentation model is generated using contouring by a set of clinicians of a clinic, the user being a member of the clinic.

14. The at least one machine-readable medium of claim 10, wherein the operations further cause the processing circuitry to send, to the user, updated slices from the set of contoured structures for iteratively adjusting the updated slices.

15. The at least one machine-readable medium of claim 10, wherein the operations further cause the processing circuitry to receive, during a clinical workflow, a user edit to a segmentation generated by the personalized segmentation model, and use the segmentation to iteratively update the personalized segmentation model via the transfer learning technique.

16. The at least one machine-readable medium of claim 10, wherein the adjusted selected slices include ten or fewer slices.

17. The at least one machine-readable medium of claim 10, wherein the initial segmentation model and the personalized segmentation model are specific to a particular disease or organ.

18. The at least one machine-readable medium of claim 10, wherein the structure model is trained to convert two-dimensional slices to three-dimensional structures.

19. A system comprising:
processing circuitry; and
memory, including instructions, which when executed by the processing circuitry, cause the processing circuitry to:
send, to a user, an initial segmentation model, trained using a first set of planning image structures, and selected slices from a second set of planning image structures;
receive, from the user, adjusted selected slices, the adjusted selected slices being modified by the user;
generate, using the adjusted selected slices as an input to a structure model, a set of contoured structures; and
modify the initial segmentation model using the set of contoured structures via a transfer learning technique to output a personalized segmentation model for the user.

20. The system of claim 19, wherein the initial segmentation model and the personalized segmentation model are specific to a particular disease or organ.
